(19) United States
(12) Patent Application Publication
(10) Pub. No.: US 2025/0349394 A1
(43) Pub. Date: Nov. 13, 2025

(54) SYSTEM AND METHOD FOR SPECTROSCOPIC DETERMINATION OF CHEMICAL COMPOSITIONS FROM SAMPLE SCANS

(71) Applicant: THERMO SCIENTIFIC PORTABLE ANALYTICAL INSTRUMENTS INC., Tewksbury, MA (US)

(72) Inventors: Emily Berman, Tewksbury, MA (US); Ting Yasuhara, Tewksbury, MA (US)

(21) Appl. No.: 19/201,617

(22) Filed: May 7, 2025

Related U.S. Application Data
(60) Provisional application No. 63/644,023, filed on May 8, 2024.

(51) Int. Cl. G16C 20/20; G01N 21/65; G01N 33/15

(52) U.S. Cl. CPC G16C 20/20; G01N 21/65; G01N 33/15; G01N 2201/129

(57) ABSTRACT

Systems and methods for spectroscopic determination of chemical compositions from sample scans. One method includes receiving a first user selection input representative of a user selection of any one or one or more scan modes and receiving a second user selection input representative of a user selection of any one of the one or more target chemical substances to identify. The method also includes receiving scan data generated by a scan of the sample of the unknown chemical composition, and determining a result based on the received scan data generated by the scan of the sample of the unknown chemical composition. The method further includes comparing the result against an expected result for a sample scan associated with the selected target chemical substance and generating instructions for displaying indicia representative of a primary compound associated with the selected target chemical substance.

### RELATED APPLICATIONS

This application claims priority to U.S. Provisional Application No. 63/644,023, filed May 8, 2024, the entire content of which is incorporated by reference herein.

### FIELD

The present disclosure generally relates to systems and methods for conducting spectroscopic analytical techniques, such as Raman spectroscopy. In particular, systems and methods are disclosed for determining a result based on scan data generated by a scan of a sample of an unknown chemical composition.

### BACKGROUND

The interception of counterfeit drug products, such as pills including fentanyl, has become a severe policing problem worldwide. It is sometimes difficult to identify counterfeit pills because fentanyl, when present, may be present in trace amounts, thereby making it difficult to directly detect fentanyl. This can lead to false arrests or releasing suspects who may indeed be in position of counterfeit drug products. While a properly equipped lab can make a definitive analysis, typical lab equipment does not lend itself to use by law enforcement personnel in the field because it is either too heavy, cumbersome, difficult to operate, or too expensive to distribute widely to large numbers of law enforcement personnel.

### SUMMARY

According to one aspect of the present disclosure, a computer-implemented method on an analytical instrument support apparatus is disclosed. The method includes receiving, by one or more processors, a first user selection input representative of a user selection of any one of one or more scan modes. In response to receiving the first user selection input, instructions are generated by one or more processors for displaying to the user one or more selection options for any one of one or more target chemical substances to identify via a sample scan. A second user selection input is received by one or more processors that is representative of a user selection of any one of the one or more target chemical substances to identify. In response to receiving the second user selection input, instructions are generated by one or more processors for initiating a laser to scan a sample of an unknown chemical composition. The laser is communicatively connected to the analytical instrument support apparatus. Scan data is received by one or more processors by a scan of the sample of the unknown chemical substance. A result is determined by one or more processors based on the received scan data generated by the scan of the sample of the unknown chemical substance. In response to determining the result, the result is compared by one or more processors against an expected result for a sample scan associated with the selected target chemical substance. In response to the result based on the scan of the sample of the unknown substance matching the expected result for the sample scan associated with the selected target chemical substance, instructions are generated by one or more processors for displaying indicia representative of a primary compound associated with the selected target chemical substance. In response to the result based on the scan of the sample of the unknown chemical substance not matching the expected result for the sample scan associated with the selected target chemical substance, instructions are generated by one or more processors for displaying indicia to the user representing that the scan of the sample of the unknown chemical substance result was inconclusive.

According to another aspect of the present disclosure, an analytical instrument support system is disclosed. The analytical instrument support system includes one or more processors, one or more non-transitory computer-readable storage media, and program instructions stored on at least one of the one or more non-transitory computer readable storage media for execution by at least one of the one or more processors. The program instructions comprise: (i) program instructions to receive a first user selection input representative of a user selection of any one of one or more scan modes; (ii) in response to receiving the first user selection input, program instructions for displaying to the user one or more selection options for any one of one or more target chemical substances to identify via a sample scan; (iii) program instructions to receive a second user selection input representative of a user selection of any one of the one or more target chemical substances to identify; (iv) in response to receiving the second user selection input, program instructions for initiating a laser to scan a sample of an unknown chemical composition, the laser being communicatively connected to the analytical instrument support system; (v) program instructions to receive scan data generated by a scan of the sample of the unknown chemical composition; (vi) program instructions to determine a result based on the received scan data generated by the scan of the sample of the unknown chemical composition; (vii) in response to determining the result, program instructions to compare the result against an expected result for a sample scan associated with the selected target chemical substance; (viii) in response to the result based on the scan of the sample of the unknown chemical composition matching the expected result for the sample scan associated with the selected target chemical substance, program instructions for displaying indicia representative of a primary compound associated with the selected target chemical substance; and (ix) in response to the result based on the scan of the sample of the unknown chemical composition not matching the expected result for the sample scan associated with the selected target chemical substance, program instructions for displaying indicia to the user representing that the scan of the sample of the unknown chemical composition result was inconclusive.

According to another aspect of the present disclosure, an analytical instrument is disclosed. The analytical instrument includes a light source configured to emit light toward a surface of a sample, a spectrograph configured to acquire a Raman spectrum from the surface of the sample in response to the emitted excitation light, one or more processors, one or more non-transitory computer-readable storage media, and program instructions stored on at least one of the one or more non-transitory computer-readable storage media for execution by at least one of the one or more processors. Execution of the program instructions by at least one of the one or more processors cause the analytical instrument to implement the following acts, comprising: (i) program instructions to receive a first user selection input representative of a user selection of any one of the one or more scan modes; (ii) in response to receiving the first user selection input, program instructions for displaying to the user one or more selection options for any one of one or more target chemical substances to identify via a sample scan; (iii) program instructions to receive a second user selection input representative of a user selection of any one of the one or more target chemical substances to identify; (iv) in response to receiving the second user selection input, program instructions for initiating a laser to scan a sample of an unknown chemical composition, the laser being communicatively connected to the analytical instrument support system; (v) program instructions to receive scan data generated by a scan of the sample of the unknown chemical composition; (vi) program instructions to determine a result based on the received scan data generated by the scan of the sample of the unknown chemical composition; (vii) in response to determining the result, program instructions to compare the result against an expected result for a sample scan associated with the selected target chemical substance; (viii) in response to the result based on the scan of the sample of the unknown chemical composition matching the expected result for the sample scan associated with the selected target chemical substance, program instructions for displaying indicia representative of a primary compound associated with the selected target chemical substance; (ix) in response to the result based on the scan of the sample of the unknown chemical composition not matching the expected result for the sample scan associated with the selected target chemical substance, program instructions for displaying indicia to the user representing that the scan of the sample of the unknown chemical composition result was inconclusive.

There is no specific requirement that a system, method, or technique relating to determination-based spectroscopy include all of the details characterized herein, in order to obtain some benefit according to the present disclosure. Thus, the specific examples characterized herein are meant to be exemplary applications of the techniques described, and alternatives are possible.

### BRIEF DESCRIPTION OF THE DRAWINGS

Features and advantages of the present technology will become more apparent from the following detailed description of exemplary embodiments thereof taken in conjunction with the accompanying drawings in which:

FIG. 1 is a block diagram of an exemplary analysis system, according to some implementations of the present disclosure.

FIG. 2 illustrates an optical architecture for a spectrometer included in the analysis system of FIG. 1, according to some implementations of the present disclosure.

FIG. 3 illustrates another optical architecture for the spectrometer included in the analysis system of FIG. 1, according to some implementations of the present disclosure.

FIG. 4 illustrates a further optical architecture for the spectrometer included in the analysis system of FIG. 1, according to some implementations of the present disclosure.

FIG. 5 illustrates yet another optical architecture for the spectrometer included in the analysis system of FIG. 1, according to some implementations of the present disclosure.

FIG. 6 is a flowchart of an exemplary process to determine a result from a scan of an unknown chemical composition, according to some implementations of the present disclosure.

FIG. 7 illustrates a graphical user interface (GUI) of a first selection of any one of one or more scan modes, according to some implementations of the present disclosure.

FIG. 8A illustrate a GUI of a second selection of any one of one or more exemplary target chemical substances to identify via a sample scan, according to some implementations of the present disclosure.

FIG. 8B illustrates a GUI to initiate a scan of an unknown chemical composition associated with the selection of a target chemical substance, according to some implementations of the present disclosure.

FIGS. 9A and 9B illustrate GUIs of a scan of a sample of an unknown chemical composition, according to some implementations of the present disclosure.

FIG. 10 illustrates a GUI of an exemplary result of a scan of an unknown chemical composition where the target chemical substance is detected, according to some implementations of the present disclosure.

FIG. 11 illustrates a GUI of another exemplary result of a scan of an unknown chemical composition where the target chemical substance is not detected, according to some implementations of the present disclosure.

While the present technology is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Unless otherwise defined, all technical and scientific terms used herein have the same meaning as commonly understood by one of ordinary skill in the art. In case of conflict, the present document, including definitions, will control. Example methods and systems are described below, although methods and systems similar or equivalent to those described herein can be used in practice or testing of the present disclosure. All publications, patent applications, patents and other references mentioned herein are incorporated by reference in their entirety. The systems, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Raman measurement” refers to a Raman system where the illumination spot diameter remains fixed-size and has a uniform radial distribution.

“Aspheric diffuse ring producing optic” refers to various implementations for producing the distributed spot which includes an aspheric diffuse ring producing optic, or ADRPO. In some implementations, aspheric optics may include what is referred to as an axicon or conical optic which produces a ring of intensity but has higher order aspheric terms to produce the spread-out pattern. In some implementations, the aspheric optic may have coefficients of A1=0.01, A2=0.06, and A4=0.002, with all other terms being zero.

“Collimating lens” refers to optical elements that transform the incoming light direction to parallel paths.

“Filter” refers to optical elements that remove some wavelengths of incoming light.

“Focusing optics” refers to optical elements that transform the incoming light direction to a point in space.

“Light source” refers to a light source used for excitation in spectroscopy application. Exemplary systems and methods may include a laser that is adapted for Raman spectroscopy such as 785 m, or 1064 nm. Exemplary light sources could also include a broad band source such as an LED.

“Sample surface plane” refers to the surface of the sample under test where the illumination area is directed.

“Steering mirrors” refers to optical elements used to change the direction of light path.

“Raman spectrum” refers to a spectrum of data values that may include a bright spectrum and/or a dark spectrum. Where the bright spectrum is the scattered light from the sample hitting a detector. The dark spectrum is a spectrum received when no light hits the detector. The dark spectrum captures the shape of the baseline offset.

Systems, methods and techniques are disclosed for determining a result of a scan of an unknown chemical composition based on received scan data generated by a scan from a Raman laser. The present disclosure can be particularly desirable by providing an accurate determination of whether a generated Raman signature of an unknown chemical composition contains a primary compound generally associated with a target chemical substance or if the scan of the unknown chemical composition returns an inconclusive result. For example, in some aspects, the present disclosure provides for improved methods for determining whether an unknown chemical composition contains a primary compound with a probability confidence level of greater than 95%. The present disclosure desirably provides improved methods for determining whether an unknown chemical composition is associated with a counterfeit drug product. For example, the present disclosure provides an improved method for determining that a generated Raman signature from the unknown chemical composition does not match any reference signature that is associated with a target chemical substance, where the reference signature is stored in a database or library of reference signatures. When the generated Raman signature does not match any reference signature for a target chemical substance, a determination of an inconclusive result is generated. An inconclusive result may include that the unknown chemical composition is associated with a counterfeit drug product and in some instances, the counterfeit drug product may include fentanyl.

In Raman spectroscopy, light typically from a laser and of a known wavelength (typically infrared or near infrared) is directed at a sample of an unknown chemical composition. The laser light (i.e., a Raman pump) interacts with the electron clouds in the molecules of the sample and, as a result of this interaction, experiences selected wavelength shifting. The precise nature of this wavelength shifting depends upon the compounds or substances present in the sample. A unique wavelength signature (i.e., a Raman signature) is produced by each sample compound or substance. This unique Raman signature permits the sample to be identified and characterized. More specifically, the spectrum of light returning from the sample is analyzed with a spectrometer so as to identify the Raman-induced wavelength shifting in the Raman pump light, and then this wavelength signature is compared (e.g., by a computing device) with a library of known Raman signatures, whereby to identify the nature of the sample, including the presence or absence of select target chemical compounds or substances.

As described herein, the scan data generated by a scan of the sample of the unknown chemical composition can include one or more Raman measurement parameters that are provided to an analytic instrument to determine if there is a match with known Raman signatures stored in a library of reference signatures. The Raman measurement parameters may include, for example, scan time and one or more Raman shift wavenumbers.

The system and methods described herein include operations for comparing generated Raman scan data with a library of known target chemical substance data stored on a data storage device. For example, received scan data generated based on a scan of a sample of an unknown chemical composition can be compared against known target chemical substance data, where the received scan data either matches a known target chemical substance data or does not match a known target chemical substance data.

The present disclosure is now described with reference to the drawings, wherein like reference numerals are used to refer to like elements throughout. In the following description, for purposes of explanation, numbers of specific details are set forth in order to provide an improved understanding of the present disclosure. It may be evident, however, that the systems and methods of the present disclosure may be practiced without one or more of these specific details. In other instances, well-known structures and devices are shown in block diagram form in order to facilitate describing the systems and methods of the present disclosure.

It should be understood that although implementations are described herein as being used with a spectrometer or other optical instrument, implementations can be constructed as stand-alone devices for measuring an electrochemical property of a sample compound or substance. Furthermore, although some implementations are described herein with respect to measuring an electrochemical property of a sample compound or substance, exemplary methods and systems described herein can be used to measure other electrochemical properties, such as, for example a Raman spectrum of the sample compound or substance.

Exemplary analysis systems, such as Raman spectroscopy systems, can be used in a variety of environments to identify unknown materials, to evaluate the threat posed by unknown materials, to provide positive identification of packaged raw materials, or to provide general security screening functions of a variety of substances. Exemplary analysis systems can include a wide range of sizes, from portable, handheld instruments to larger systems in permanent laboratories.

I. Exemplary Analysis systems

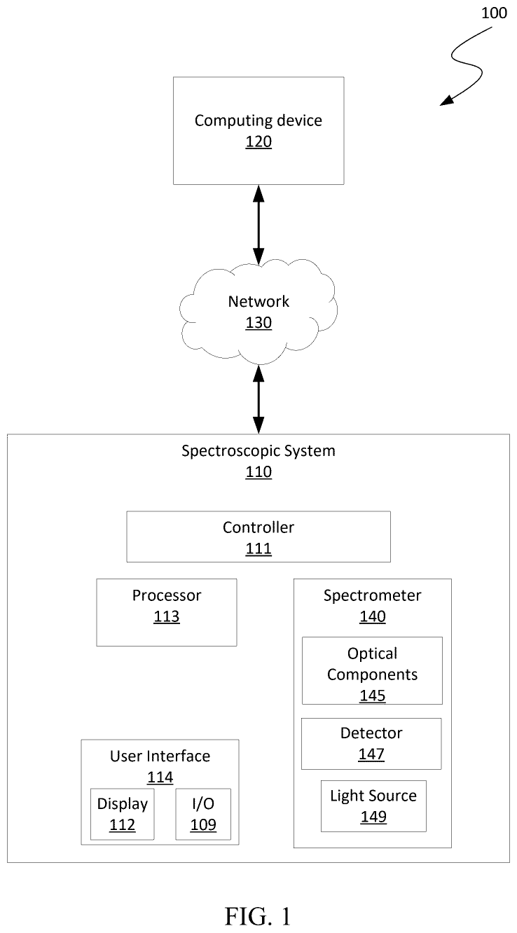

Those of ordinary skill in the art appreciate that there are a variety of different optical architectures and arrangements utilized in the field of Raman spectroscopy. FIG. 1 provides an illustrative example of an analysis system 100 (also referred to herein as “analyzer 100”) that comprises an optical architecture and other elements that operate to measure one or more Raman spectra from a sample via one or more of the methods described herein.

The analyzer 100 illustrated in FIG. 1 includes a spectroscopic system 110 communicatively coupled to a computing device 120 via a network 130. As illustrated in FIG. 1, the spectroscopic system 110 includes a controller 111, an electronic signal processor 113, and a spectrometer 140 (e.g., a Raman spectrometer).

It will be appreciated that, in some implementations, at least a portion of the computing device 120 may be located separate from the spectroscopic system 110, which provides the opportunity for increased computing power at a central location or across multiple locations. One skilled in the art can envision various interconnections, both physical and wireless, between the components of the analyzer 100. It will further be appreciated that, in some implementations, the spectroscopic system 110 and the computing device 120 may be communicatively coupled without the network 130 (e.g., via a dedicated wired or wireless connection). Alternatively, some implementations of the analyzer 100 may not require the resources of computing device 120 but may instead utilize resources internal to the spectroscopic system 110 to perform the methods described herein. Thus, computing device 120 may not be necessary for operation of the analyzer 100 and/or the spectroscopic system 110 and the example of FIG. 1 should not be considered as limiting. As described herein, the analyzer 100 may be used to measure one or more Raman spectra from a sample compound or substances via one or more of the methods described herein.

It should be understood that, in some implementations, the components of the analyzer 100 and/or the spectroscopic system 110 illustrated in FIG. 1 may be included in a common housing forming an analytical instrument that may include a benchtop or a portable Raman spectrometer device (e.g., a handheld device). However, in other implementations, one or more components of the analyzer 100 and/or the spectroscopic system 110 may be contained in separate housings or devices and may be coupled (e.g., communicatively, electrically, mechanically, or the like) as needed to carry out the methods described herein. Also, in some implementations, the operations described herein as being performed by the components of the analyzer 100 and/or the spectroscopic system 110 may be combined and distributed in various ways. For example, in some implementations, an electronic signal processor 113 may be part of a controller 111, wherein the controller 111 is configured to perform the operations of the electronic signal processor 113 as described herein. Furthermore, the operations described herein as being performed by the controller 111 may be distributed among multiple controllers. In the same or alternative examples, operations described herein as being performed by controller 111 may be distributed among one or more computing devices (e.g., the electronic signal processor 113, the computing device 120, or multiple computing devices). In some implementations, the controller 111 is configured to control operation of the spectrometer 140, wherein the electronic signal processor 113 is configured to control other components of the spectroscopic system 110 (e.g., communication with the computing device 120). However, these roles of the controller 111 and the electronic signal processor 113 may be combined and distributed in various ways, and, in some implementations, the spectroscopic system 110 includes only the controller 111 or the electronic signal processor 113 and the included devices performed the functionality of both the controller 111 and the electronic signal processor 113 as described herein.

The spectroscopic system 110 may also include additional components (such as power components), a user interface 114 (such as a display 112 and/or user input and/or output (“I/O”) device 109, such as, for example, a keyboard, a mouse, a touch screen), optical components (e.g., mirrors, lens, fiber optic cables, gratings, and filters), and the like. The spectrometer 140 included in the spectroscopic system 110 includes one or more optical components 145, a detector 147 (e.g., a CCD detector, a PMT detector, or other detector known in the art), and a light source 149. The light source 149 provides an excitation beam (e.g., excitation laser providing 785 nm or 1064 nm light) to a sample (not shown in FIG. 1).

As described above, the spectroscopic system 110 and/or the spectrometer 140 may comprises a fully integrated portable system operated by a user on battery power to take Raman spectroscopy measurements in a variety of environments, such as, for example, a laboratory setting, a manufacturing (e.g., bioreactor based) setting, a remote setting, etc. Also, in the same or alternative implementations, elements of the spectroscopic system 110 may be utilized as separated systems communicatively connected (e.g., optically, wirelessly, electrically, mechanical, and the like) operated on battery power and/or power outlets connected to a central power source to take Raman spectroscopy measurements in the variety of environments described.

Referring now to light source 149 of spectrometer 140, it will be appreciated that implementations of light source 149 may emit wavelengths of light as needed for an application, for example, including or between a range of about 400 nm to about 1064 nm, a range of about 400 nm to about 750 nm, a range of about 400 nm to about 600 nm, a range of about 400 nm to about 500 nm, a range of about 600 nm to about 900 nm, a range of about 700 nm to about 850 nm, a range of 600 nm to 1064 nm, a range of 750 nm to 1064 nm, a range of 850 nm to 1064 nm, a range of 950 nm to 1064 nm, as well as a wavelength of about 785 nm, or a wavelength of about 1064 nm.

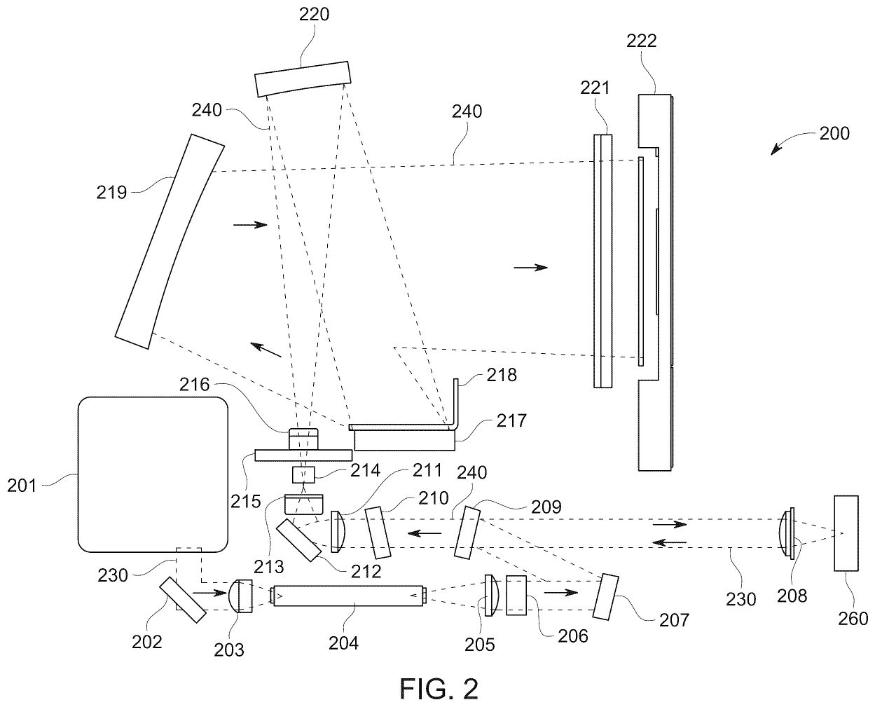

FIG. 2 provides an illustrative example of one implementation of an optical architecture comprising optical components of the spectrometer 140 (see FIG. 1), that are otherwise collectively referred to herein as an optical system 200. It will be appreciated that different optical architectures of Raman spectrometer are known in the art and thus the example of FIG. 2 should not be considered as limiting. For example, some implementations employ what are referred to as transmission gratings rather the reflection gratings, as well as associated differences in optical architecture.

The example of FIG. 2 illustrates one implementation of light source 149 (see FIG. 1) as laser assembly 201 comprising a laser source that produces a beam of light that travels along optical or beam path 230 (e.g., arrows illustrate direction of travel of the light beam) to sample 260. It will be appreciated that sample 260 may include any type of sample of interest to a user and may include substantially dry samples (e.g., a powder, solid material), substantially fluid samples (e.g., a liquid, gas), or some combination thereof (e.g., a gel). In response to the light from laser assembly 201, the sample 260 produces scattered light (e.g., comprising a Raman portion and a Rayleigh portion of scattered light), which travels along beam path 240.

In some implementations, the laser assembly 201 may produce laser power as needed for an application for example, including or between a range of about 250 mW to about 750 mW; about 250 mW to about 700 mW; about 250 mW to about 650 mW; about 250 mW to about 600 mW; about 250 mW to about 550 mW; about 250 mW to about 500 mW; about 250 mW to about 450 mW; about 250 mW to about 400 mW; about 250 mW to about 350 mW; about 250 mW to about 300 mW; or about 250 mW. Also in some implementations, the laser power affects the values of the base value and the bright-max intensity values when sample 260 is scanned. It will be appreciated that other ranges and/or levels of laser power are known in the art and thus the example described for laser assembly 201 should not be considered as limiting.

FIG. 2 also illustrates one implementation of an architecture that directionally controls the beam path 230 and the beam path 240 as well as conditions one or more characteristics of the beam of light produced from the laser assembly 201 as well as from the sample 260. For example, a turning mirror 202 redirects beam path 230 to focusing lens 203 that focuses the beam onto a waveguide phase scrambler 204 (e.g., to adjust the phase characteristics of the beam). The beam exits waveguide phase scrambler 204 and travels to a collimating lens 205 (e.g., which adjusts collimation characteristics of the beam), then to a broadband filter 206 transmissive to a specific wavelength or range of wavelengths of light. The beam travels to a flat mirror 207 that redirects the beam path 230 to a selective element 209. It will be appreciated that the selective element 209 may include a dichroic mirror, a notch filter, or other element that comprises substantially reflective characteristics to the wavelength(s) of the beam from laser assembly 201 and comprises substantially transmissive characteristics to a wavelength or wavelength range associated with Raman scattered light from sample 260. In the described example, selective element 209 redirects the beam path 230 to a lens 208 that focuses the beam to the sample 260. In the described example, the lens 208 may include any type of lens known in the art such as an objective lens that focuses the beam onto the sample 260. Also, some implementations of the lens 208 comprise special configurations and characteristics that provide advantages for different types of the sample 260 as will be described below.

The lens 208 collects Raman scattered light and Rayleigh scattered light produced from the sample 260 in response to the beam from the laser assembly 201 and produces the beam path 240 that travels back to the selective element 209 and a second selective element 210. As described above, the selective elements 209 and 210 are substantially transmissive to the wavelengths of the Raman scattered light, allowing the beam path 240 to pass through to additional optical elements that further adjust the path and conditions the characteristics of the beam traveling along the beam path 240. For example, the optical elements may include a focusing lens 211, a flat mirror 212, a baffle 213, a slit 214, a baffle 215, and a collimating lens 216.

The beam path 240 travels from the collimating lens 216 to a mirror 220 that reflects the beam path 240 toward a diffraction grating 217. It will be appreciated that, in the example of FIG. 2, the diffraction grating 217 comprises a reflective diffraction grating that produces a spectral distribution of light. The beam path 240 then travels to a focusing mirror 219 that redirects the beam path 240 to a focusing lens 221 that directs the beam to elements of a detector 222 (one implementation of the detector 147 of FIG. 1). It will also be appreciated that FIG. 2 illustrates a baffle 218 that, in some implementations, controls stray light.

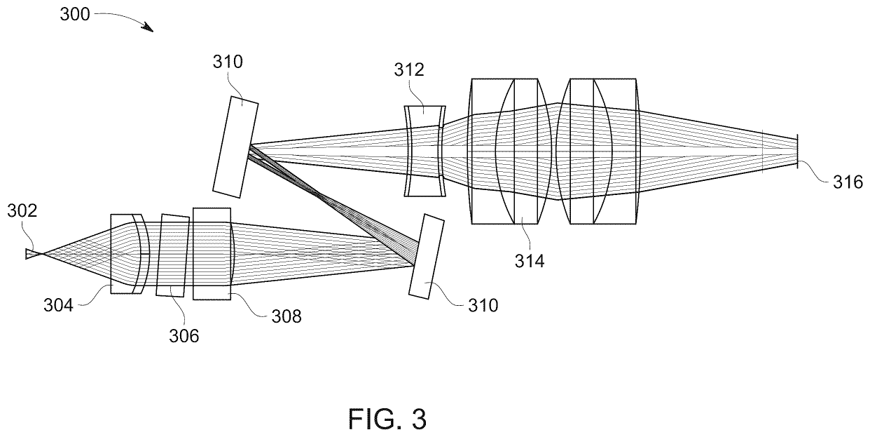

As described above, it will be appreciated that a variety of implementations of lens 208 are available that provide different focusing and light collection characteristics. For example, FIG. 3 provides an example implementation of an optical architecture useful for analyzing a sample contained in a package (e.g., a bag, bottle, etc.), where the optical architecture comprises some components of the optical system 200 (see FIG. 2) and other components that provide the characteristics of lens 208 (see FIG. 2), collectively referred to as an optical arrangement 300. In the described example, the optical arrangement 300 includes an element 302 that may include a focusing lens 203 (see FIG. 2) or an output from an optical fiber. Element 302 directs a beam (e.g., produced from light source 149 or laser assembly 201 or a Raman laser 119—see FIGS. 1, 2, and 5) to a collimating lens 304 that produces a substantially collimated beam. In the described example, the collimating lens 304 can be movably mounted such that it can change position along the axis of the optical path. The range of motion includes a range of about 0.1 mm to about 10 mm to allow for a change in spot size on the sample surface to range from about 10 microns to about 10 mm. It will also be appreciated that in some implementations any of the collimating lens 304, a concave focusing lens 312, and/or focusing optics 314, either alone or in combination, may be movably mounted to effect a change in spot size.

The collimating lens 304 directs the substantially collimated beam into an aspheric diffuse ring producing optic 308 configured to produce a light pattern that is radially diffuse. The intensity of the output from the aspheric diffuse ring producing optic 308 is more intense at the outer edge of the resulting pattern than in the center. While this pattern could be projected directly onto a sample surface 316, in practical application it is advantageous to use one or more steering mirrors 310, one or more filters 306, and focusing elements, such as, for example, a concave focusing lens 312 and focusing optics 314, to direct the radially diffuse light pattern onto the sample surface 316.

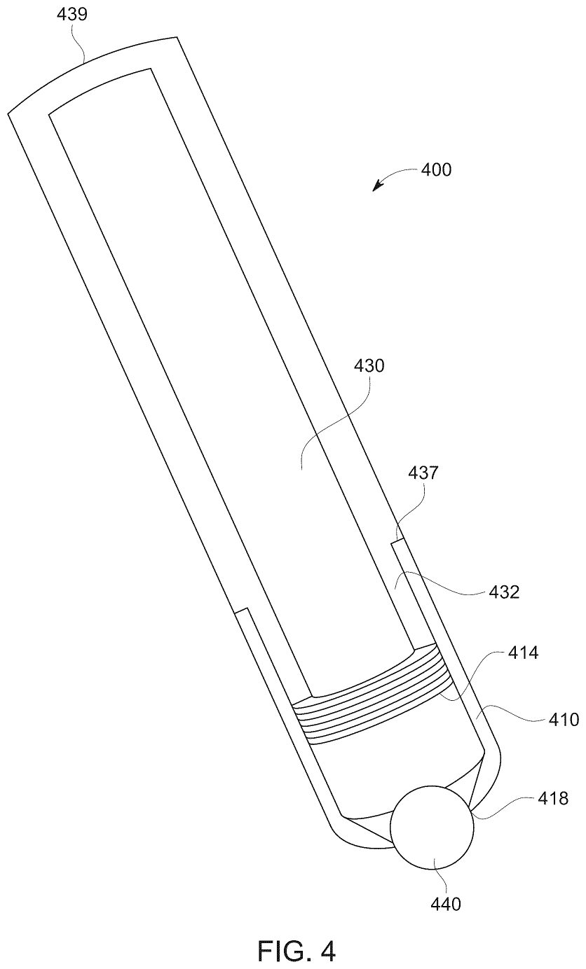

FIG. 4 provides an example of another implementation of the lens 208 (see FIG. 2), wherein this example may be useful for analyzing a fluid or semi-fluid sample. The implementation illustrated in FIG. 4 comprises some components of the optical system 200 and other components that provide characteristics of what is generally referred to as an “immersion probe,” wherein the components are collectively referred herein to as an optical arrangement 400. The implementation illustrated in FIG. 4 comprises a spherical lens 440 (referred to herein as “lens 440”) seated within a cylindrical probe tip 410 (referred to herein as “probe tip 410”) at lens opening 418. A seal between the probe tip 410 and the lens 440 is formed at the opening by any means known in the art, including all forms of welding or braising and the use of epoxies or other adhesives. The probe tip 410 may be any length. Optionally, the probe tip 410 may have threads 414 on its interior surface and may be extended using probe tube 430, which has threaded collar 432 for threading into probe tip 410. A seal is optionally formed between probe tube lip 437 and the distal end of probe tip 410. Further, in the described example, the optical arrangement 400 includes fiber optic coupling 439 that transmits illumination light from the laser assembly 201 (see FIG. 2) as well as scattered light from the sample 260 (see FIG. 2), wherein the sample 260 may include a liquid sample where lens 440 is immersed in the liquid. Also in the described example, the optical arrangement 400 may be configured as a separated element from spectroscopic system 110 (see FIG. 1) where an optical fiber provides optical communication between spectroscopic system 110 and the optical arrangement 400.

It will be appreciated that the examples provided in FIG. 3 and FIG. 4 are for the purposes of illustration and some implementations may include additional or fewer elements as needed for an application. For instance, in some implementations one or more windows, collimating lenses or other optical elements may be employed in applications that utilize a fiber optic coupling or other need for conditioning a beam or protecting internal environments. Therefore, the examples provided in FIG. 3 and FIG. 4 should not be considered as limiting.

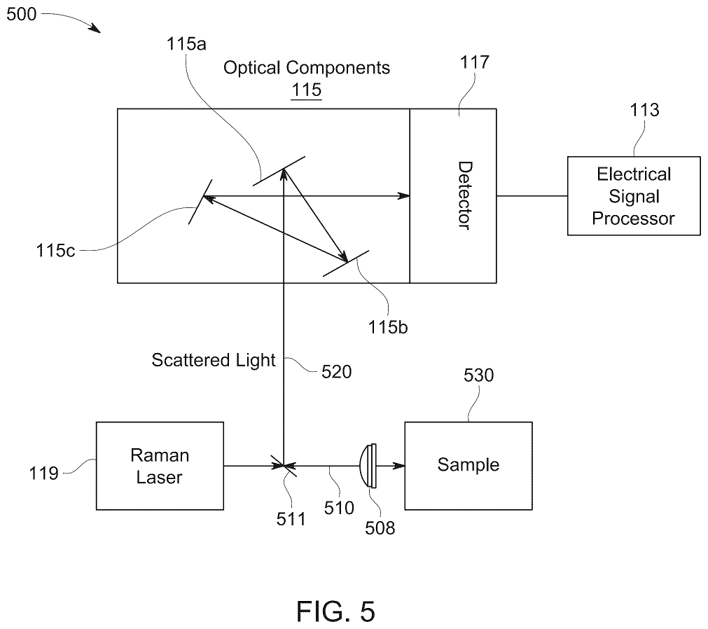

FIG. 5 provides another example of an implementation of an optical architecture comprising optical components of the spectrometer 140 (see FIG. 1), that are otherwise collectively referred to herein as the optical system 500. It will be appreciated that different optical architectures of Raman spectrometer are known in the art and thus the example of FIG. 5, similar to the examples of FIGS. 1 to 4, should not be considered as limiting.

The example of FIG. 5 illustrates one implementation of the light source 149 (see FIG. 1) as a Raman laser 119 comprising a laser source that produces a beam of light that travels along a first optical or beam path 510 (e.g., arrows illustrate direction of travel of the light beam) to a sample 530. Like sample 260 (see FIG. 2), it will be appreciated that sample 530 may include any type of sample of interest to a user which may include substantially dry samples (e.g., a powder, solid material), substantially fluid samples (e.g., a liquid, gas), or some combination thereof (e.g., a gel). In response to the light from the Raman laser 119, the sample 530 produces scattered light along a second optical or beam path 520 (e.g., comprising a Raman portion and a Rayleigh portion of scattered light).

In some implementations, the Raman laser 119 may produce laser power as needed for an application for example, including or between a range of about 250 mW to about 1050 mW, including various subranges therebetween such as the non-limiting subranges described above for the light source 149 and the laser assembly 201. It will also be appreciated that in some implementations, the laser power affects the values of the base value and the bright-max intensity values when the sample 530 is scanned.

FIG. 5 illustrates an architecture that in some implementations directionally controls the first beam path 510 and/or the second beam path 520. In some implementations, the beam paths 510, 520 can be controlled using one or more of turning mirrors, waveguide phase scramblers, various lenses, broadband filters, or selective elements (e.g., mirrors, notch filters, or other elements with substantially reflective characteristics to the wavelength(s) of the beam from the Raman laser 119 and/or substantially transmissive characteristics to a wavelength or wavelength range associated with Raman scattered light from sample 530). In the described example, a selective element 511 is transmissive to the laser wavelengths emitted from the Raman laser 119 allowing the first beam path 510 to be directed to a lens 508 that focuses the beam onto the sample 530. In the described example, the lens 508 may include any type of lens known in the art such as an objective lens or lens architecture such as used in the optional arrangements 300 or 400 (see FIG. 3 and FIG. 4) that focuses the beam onto the sample 530.

Some implementations of the lens 508 include special configurations and characteristics that provides advantages for different types of samples. For example, the lens 508 can collect Raman scattered light and Rayleigh scattered light produced from the sample 530 in response to the beam from the Raman laser 119. The scattered light collected by the lens 508 is directed back from the surface of the sample 530 and travels back along the first beam path 510 to the selective element 511 (e.g., a beam splitter, such as, for example, a dichroic mirror) that directs the scattered light along the second beam path 520. In some implementations, the selective element 511 is substantially reflective to the wavelengths of the Raman scattered light, allowing the second beam path 520 to be directed to additional optical elements that further adjust the path and condition the characteristics of the beam traveling along the second beam path 520. Other optical arrangements are also contemplated for the selective element 511 for directing the scattered light along the second beam path 520.

As illustrated in FIG. 5, the optical system 500 also includes one or more optical components 115 (also referred herein as optical components 115a-115c), which can include one or more of collimating lens and mirrors, filters, such as, for example, a notch filter, diffraction gratings, and/or mirror relays. The scattered light is directed by one or more of optical components 115a-115c onto a detector 117 (an implementation of the detector 147 of FIG. 1). Signal processing and/or digitizing of signals associated with the scattered light that is received by the detector 117 is performed by an electronic signal processor associated with optical system 500, which may be, for example, the electronic signal processor 113, the controller 111, the computing device 120, or a combination thereof. For example, in some implementations, the electronic signal processor 113 may be a suitably programmed microprocessor or application specific integrated circuit including a read-only or read-write memory of any known type which holds instructions and data for spectrometer operation as described herein.

As described above, it will be appreciated that a variety of implementations of the lens 508 are available that provide different focusing and light collection characteristics.

Returning to FIG. 1, the computing device 120 may be a standalone device, a server, internet of things (IoT), a laptop computer, a tablet computer, a netbook computer, a personal computer (PC), a smartphone, a personal digital assistant (PDA), a desktop computer, or any programmable electronic device capable of receiving, sending, and processing data. In some implementations, the computing device 120 includes one or more processors, one or more input/output processors, and one or more memory or data storage devices. In some implementations, the computing device 120 also includes one or more input/output devices, such as, for example, a display, a touchscreen, a keyboard, a mouse, or the like, which may be used to provide calibration or setting options to a user for operating the spectroscopic system, to provide analysis results to a user, or a combination thereof.

Similarly, the controller 111 (see FIG. 1) may include an electronic processor, an input/output (I/O) interface, and a data storage device (not shown); however, it should be understood that the controller 111 may have additional or fewer components. The controller 111 is suitable for the application and setting, and can include, for example, multiple electronic processors, multiple I/O interfaces, multiple data storage devices, or combinations thereof. In some implementations, some or all of the components included in the controller 111 may be attached to one or more mother boards and enclosed in a housing (e.g., including plastic, metal and/or other materials). In some implementations, some of these components may be fabricated onto a single system-on-a-chip, or SoC (e.g., an SoC may include one or more processing devices and one or more storage devices).

As used herein, “processors” or “electronic processor” or “electronic signal processor” refers to any device(s) or portion(s) of a device that process electronic data from registers and/or memory to transform that electronic data that may be stored in registers and/or memory. The electronic processor included in the controller 111 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The data storage device(s) included in the controller 111 may include one or more local or remote memory devices such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some implementations, the data storage device(s) may include memory that shares a die with a processor. In such an embodiment, the memory may be used as a cache memory and may include embedded dynamic random-access memory (eDRAM) or spin transfer torque magnetic random-access memory (STT-MRAM), for example. In some implementations, the data storage device may include non-transitory computer-readable media having instructions thereon that, when executed by one or more processors (e.g., the electronic processor included in the controller 111), causes the controller 111 to store various applications and data for performing one or more of the methods described herein or portions described herein. Alternatively, or in addition, applications and data for performing one or more of the methods described herein or portions thereof may be stored and executed on the computing device 120. Also, it should be understood that each method described herein may be implemented via one application or multiple applications.

For example, one or more data storage devices (not shown) may include substance identification program, target chemical substance data, operation instruction data, or a combination thereof. It should be understood that each method described herein may be implemented via one application or multiple applications.

A substance identification program may include one or more algorithms for determining whether one or more Raman measurement parameters associated with a result of a sample of an unknown chemical composition matches one or more Raman measurement parameters associated with one or more target chemical compounds or substances stored as part of target chemical substance data, as described below. In some implementations, substance identification program determines that the result of the sample of the unknown chemical composition matches at least one target chemical substance associated with the target chemical substance data based on, at least, a similarity measure greater than 95%, as described below, in equation (1). In some implementations, substance identification program determines that the result of the sample of the unknown chemical composition matches at least one target chemical substance based on a similarity measure greater than 95%; greater than 96%; greater than 97%; greater than 98%; or greater than 99%. In some implementations, substance identification program determines that the result of the sample of the unknown chemical composition does not match at least one target chemical substance associated with the target chemical substance data based on, at least, a similarity measure equal to or less than 95%.

The comparison between the result of the unknown chemical composition and the one or more target chemical substances associated with the target chemical substance data is based on, at least, equation (1), shown below:

$${S}_{i} = f ( {y}_{lib , i} , {y}_{meas} , {\sum }_{i} , {\sum }_{meas} , Ψ ) \qquad ( 1 )$$

Where Si is the similarity measure between (i) the ith library spectrum, y_{lib,i}, for a given library material 1 and (ii) the measure spectrum yₘₑₐₛ. A similarity metric is conditional on Σᵢ, Σₘₑₐₛ, which are representations of the “precision state” of the library (Σᵢ) and the measured spectrum (Σₘₑₐₛ) under the circumstances, and I codifies other information available at the time of the similarity analysis.

The precision-state-based similarity measurement is based on, at least, equation (2), as shown below:

$${\sum }_{m e a s} = f ( {I}_{Ral} , {I}_{Ram} , {I}_{fl} , {I}_{ambient} , {I}_{dark} , {σ}_{read} , {D}_{CCD} , {G}_{CCD} , C , T , H , t , L ) \qquad ( 2 )$$

I_{Ral} is the Raleigh scatter intensity, I_{Ram}, is the Raman scatter intensity, I_{fl} is the fluorescence intensity and I_{ambient} is the ambient light intensity. All of the parameters affect the uncertainly of the analytical measurement because they each contribute photon shot noise. I_{dark} is the dark current intensity in the CCD, the spontaneous accumulation of detector counts without impinging photons, which also contributes shot noise. Σ_{read} is the read noise, Q is quantization error, D_{CCD} is a parameter relating to variability that is a consequence of defects in the CCD construction, G_{CCD} is the gain on the CCD, T and H are the temperature and humidity conditions of the measurement, t is the time spent integrating the signals, C is physiochemical effects that can alter the exact Raman intensities of the sample of the unknown chemical composition, L is a “long-term” variability term that reflects changes in the system performance over a time period greater than that of any individual sample measurement, e.g., calibration related variability.

From the measured spectrum associated with the library (Σᵢ), the measure spectrum yₘₑₐₛ may be determined based on the unknown chemical composition is based on, at least, equation (3), as shown below:

$$\begin{matrix}{y}_{meas} = {β}_{0} + {β}_{1} * {y}_{lib , i} + e \\ = [ 1 {y}_{lib , i} ] [ \begin{matrix}{β}_{0} \\ {β}_{1}\end{matrix} ] + e \\ = {Y}_{i} β + e\end{matrix} \qquad ( 3 )$$

β₀ and β₁ are constant and multiplicative parameters (assembled into a vector β) and e is a realization of the variability in the measurement of y_{lib,i}, with distribution e˜n(0, Σᵢ). The precision state-based similarity measure can be determined by estimating the generalized lack of fit from the equation (4), as shown below:

$${\hat{e}}_{i} = ( {l}_{n} - {Y ( {Y}^{T} {\sum }_{i}^{- 1} Y )}^{- 1} {Y}^{T} {\sum }_{i}^{- 1} ) {y}_{meas} \qquad ( 4 )$$

From, equation (1), the similarity measure may be determined where the similarity measure is greater than 95%, a match may be deemed to have occurred between a result of a scan of an unknown chemical composition and at least one target chemical substance associated with the target chemical substance data. In some implementations, from equation (1), the similarity measure may be determined where the similarity measure is greater than 95%; greater than 96%; greater than 97%; greater than 98%; or greater than 99%, a match may be deemed to have occurred between a result of a scan of an unknown chemical composition and at least one target chemical substance associated with the target chemical substance data. From equation (1), a similarity measure of equal to or less than 95%, a match may not be deemed to have occurred between a result of a scan of an unknown chemical composition and at least one target chemical substance associated with the target chemical substance data.

Target chemical substance data may include one or more known measurement parameters associated with one or more target chemical substances. In some implementations, target chemical substance data includes, at least, scan time and Raman shift wavenumbers associated with one or more target chemical substances. As will be discussed in greater detail below, in some implementations the one or more target chemical substances may include opioids, stimulants, depressants, designer drugs, hallucinogens, heroin, inhalants, cannabis, methamphetamine, steroids, synthetic variants, or any combinations thereof.

In some implementations, the operation instruction data for an analytical instrument may include any number of generated instructions for a user to properly and safely handle the analysis system 100. In some implementations, operation instruction data may include generated safety instructions for a user to wear personal protective equipment (PPE) while handling and/or measuring the unknown chemical composition. In some implementations, operation instruction data may include generated safety instructions for a user to avoid eye contact with the light source (e.g., laser assembly 201 (FIG. 2) or Raman laser 119 (FIG. 5)) with the user and/or others. In some implementations, operation instruction data may include one or more operational instructions for proper use of the analysis system 100 including, for example, when the analysis system 100 is a handheld or portable system, instructions for aiming the light source toward the unknown chemical composition (e.g., aligning light emitted by the light source and output by the system 100 with the sample); positioning the system 100 (e.g., the light source) within a specified distance from the unknown chemical composition; and maintaining the aiming and positioning of the system 100 (e.g., the light source) until a result is determined based on the received scan data generated by the scan of the sample of the unknown chemical composition. The I/O interface of the controller 111 may include one or more communication chips, connectors, and/or other hardware and software to govern communications between the controller 111 and other components.

The I/O interface may include interface circuitry for coupling to the one or more components using any suitable interface (e.g., a Universal Serial Bus (USB) interface, a High-Definition Multimedia Interface (HDMI) interface, a Controller Area Network (CAN) interface, a serial Peripheral Interface (SPI) interface, an Ethernet interface, a wireless interface, or any other appropriate interface). For example, I/O interface may include circuitry for managing wireless communications for the transfer of data to and from the controller 111. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although, in some implementations the associated devices might not. Circuitry included in the I/O interface for managing wireless communications may implement any of a number of wireless standards or protocols, including but not limited to Institute for Electrical Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some implementations, circuitry included in the I/O interface for managing wireless communications may operate in accordance with a Global System for Mobile Communication (GSM), General Packet Radio Service (GPRS), Universal Mobile Telecommunications System (UMTS), High Speed Packet Access (HSPA), Evolved HPS (E-HPSA), or LTE network. In some implementations, circuitry included in the I/O interface for managing wireless communications may operate in accordance with Enhanced data for GSM Evolution (EDGE), GSM EDGE Radio Access Network (GERAN), Universal Terrestrial Radio Access Network (UTRAN), or Evolved UTRAN (E-UTRAN). In some implementations, circuitry included in the I/O interface for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some implementations, the I/O interface may include one or more antennas (e.g., one or more antenna arrays) for receipt and/or transmission of wire communications.

In some implementations, the analysis system 100 provides a stand-alone or dedicated analytical instrument or device (or set of instruments or devices) configured to perform a short scan and analysis of a sample of an unknown chemical composition. However, in other implementations, the analysis system 100 may be configured to perform additional scans or analysis of one or more samples. Combining such scanning abilities or analysis in one system (e.g., one analytical instrument) creates desirable efficiencies and improved accuracy in the analysis as multiple scans can be taken from the sample without having to change the position of the sample, reconfigure the analytical instrument, or use a separate scan for additional samples combined with the target sample, all of which can introduce delays and potentials for contamination or unintended variances between scans.

### II. Exemplary Chemical Substances

Exemplary systems and methods include scanning samples of various exemplary unknown chemical compositions that include expected target chemical substances or that include unknown chemical substances or that include known chemical substances associated with counterfeit drug products.

In some implementations, the exemplary samples of unknown chemical compositions may be in the form of any one of tablets, capsules, natural forms, liquids, loose powder, or injectables.

### A. Exemplary Target Chemical Substances

Exemplary target chemical substances may include opioids, stimulants, depressants, designer drugs, hallucinogens, heroin, inhalants, cannabis, methamphetamine, steroids, synthetic variants, or any combinations thereof.

In some implementations, exemplary target chemical substances may include a primary compound. In some instances, the primary compound is a pharmaceutical compound.

In some implementations, each of the exemplary target chemical substances has a unique Raman spectrum.

In some implementations, exemplary target chemical substances may include opioids. In some implementations, opioids may include fentanyl, hydromorphone, methadone, morphine, opium, and oxycodone.

In some implementations, exemplary target chemical substances may include stimulants. In some implementations, stimulants may include but not limited to prescription drugs such as amphetamines (e.g., Adderall® and Dexedrine®), methylphenidate (e.g., Concerta® and Ritalin®), diet aids (e.g., Didrex®, Bontril®, Preludin®, Fastin®, Adispex P®, Ionomin®, and Meridia®), and other illicitly used drugs including methamphetamine, cocaine, methcathinone, and other synthetic cathinones commonly referred to as “bath salts.”

In some implementations, exemplary target chemical substances may include depressants. In some implementations, depressants may include but not limited to barbiturates, benzodiazepines, and central nervous system (CNS) depressants. In some implementations, barbiturates may include but not limited to butalbital (e.g., Florina®), phenobarbital, Pentothal®, Seconal®, and Nembutal®. In some implementations, benzodiazepines may include but not limited to Vallium®, Xanax®, Halcion®, Ativan®, Klonopin®, Restoril®, and Rohypnol. In some implementations, central nervous system depressants may include but not limited to Lunesta®, Ambien®, Sonata®, meprobamate, methaqualone (e.g., Quallude®), and gamma-hydroxybutyrate (GHB).

In some implementations, target chemical substances may include designer drugs. In some implementations, designer drugs may include but not limited to bath salts, Flakka (alpha-PVP), and K2/spice.

In some implementations, target chemical substances may include hallucinogens. In some implementations, hallucinogens are commonly found in plants and fungi or are synthetically produced. In some implementations, hallucinogens may include ecstasy or MDMA, ketamine, LSD, peyote and mescaline, psilocybin, and salvia divinorum.

In some implementations, target chemical substances may include heroin.

In some implementations, target chemical substances may include inhalants. In some implementations, inhalants may be invisible, volatile substances found in common household products that produce chemical vapors.

In some implementations, target chemical substances may include cannabis.

In some implementations, target chemical substances may include steroids. In some implementations, steroids may include but not limited to testosterone, trenbolone, oxymetholone, methandrostenolone, nandrolone, stanozolol, boldenone, and oxandrolone.

### B. Exemplary Counterfeit Drug Products

Exemplary target chemical substances may include one or more cutting agents that may be a part of an exemplary counterfeit drug product.

Exemplary counterfeit drug products may include, among other things, fentanyl, levamisole, benzocaine, lidocaine, phenacetin, baby powder, caffeine, chloroquine, or aspirin.

In some implementations, one or more counterfeit drug products may include a target chemical substance, as discussed above, resulting in an impure, counterfeit and/or illegal drug.

In some implementations, each of the exemplary counterfeit drug products has a unique Raman spectrum. In some implementations, when exemplary systems and methods receive scan data, where one or more counterfeit drug products are present with at least one target chemical substance, the Raman spectrum associated with the one or more counterfeit drug products interferes with the unique Raman spectrum associated with the at least one target chemical substance. As a result, when the analyzer 100 receives scan data generated by the scan of an unknown chemical composition and the controller 111 cannot match the received scan data against any one of the one or more target chemical substance data stored on the target chemical substance data, the controller 111 is configured to determine that a comparison result of the scan data with the stored chemical substance data is inconclusive.

### III. Exemplary Methods of Operation

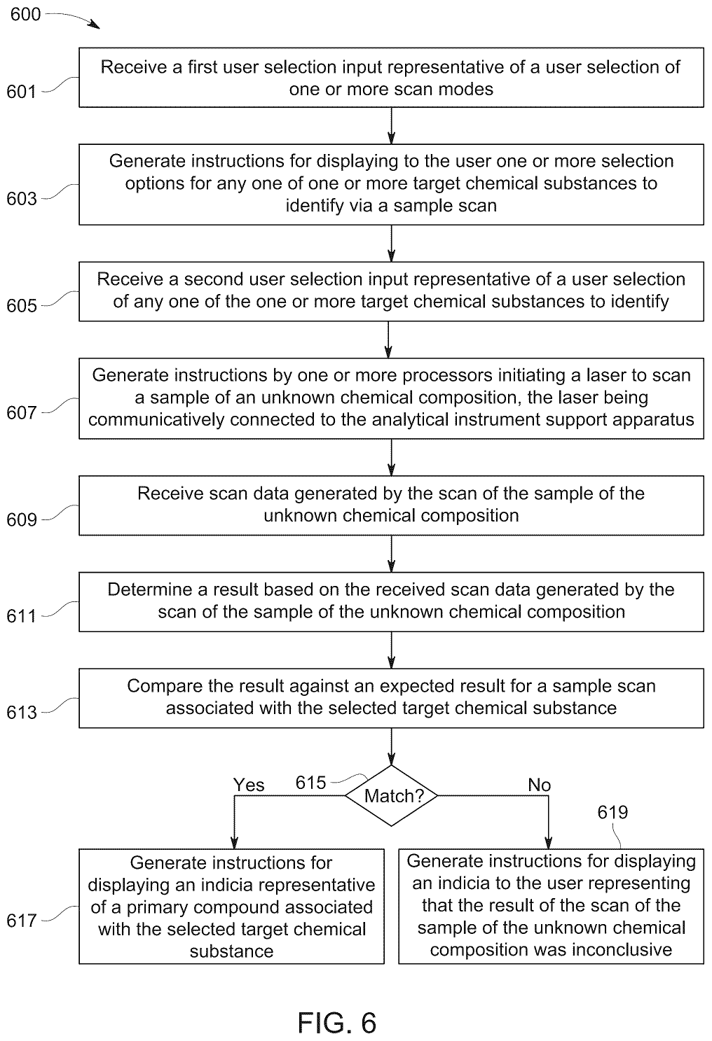

Referring now to FIG. 6, a flowchart illustrates a process 600 for determining a result from the scan of an unknown chemical composition, in accordance with some implementations of the present disclosure. Process 600 may be implemented using the spectroscopic system 110, as described above. The process 600 is described herein as being performed via the controller 111. However, it should be understood that the process 600 may be performed by one or more software and/or hardware components in various combinations and configurations. As illustrated in FIG. 6, the process 600 may include operations 601, 603, 605, 607, 609, 611, 613, 615, 617, and 619. In some implementations, the process 600 is performed in the order as illustrated in FIG. 6. For example, in some implementations, the process 600 may be performed in one or more orders other than what is illustrated in FIG. 6. For example. Operation 607 may be performed before operation 601.

In some implementations, process 600 may begin by performing scans of one or more samples, as discussed in detail above. The samples are scanned using, at least, the spectroscopic system 110, as described above in FIGS. 1-5. The spectroscopic system 110 includes a light source 149 (e.g., laser assembly 201 (FIG. 2) or Raman laser 119 (FIG. 5)) that directs a Raman laser beam (e.g., light), as described above, onto a surface or focal point of a sample. For example, using the example configuration illustrated in FIG. 5, the Raman laser 119 can be directed onto an unknown chemical composition. The resulting scattered light is directed back through the selective element 511 and the scattered light travels along the second beam path 520 and through the optical components 115 onto the detector 117. The resulting Raman spectrum of the sample is acquired by the detector 117, and signal processing and/or digitizing of the received spectrum is handled by the electrical signal processors 113.

In some implementations, a scan of the sample of the unknown chemical composition is captured from 1 millisecond (ms) to 20 seconds exposure time. In some implementations, the scan of the sample of the unknown chemical composition captures both the bright and dark Raman spectra of the sample of the unknown chemical composition.

In some implementations, the controller 111 receives scan data associated with a scan of a sample of an unknown chemical composition. In some implementations, the unknown chemical composition may be an exemplary target chemical substance, as described above. In some implementations, the sample of the unknown chemical composition may be a counterfeit drug product and may include one or more substances that are not associated with the expected product for the selected target chemical substance, as described above.

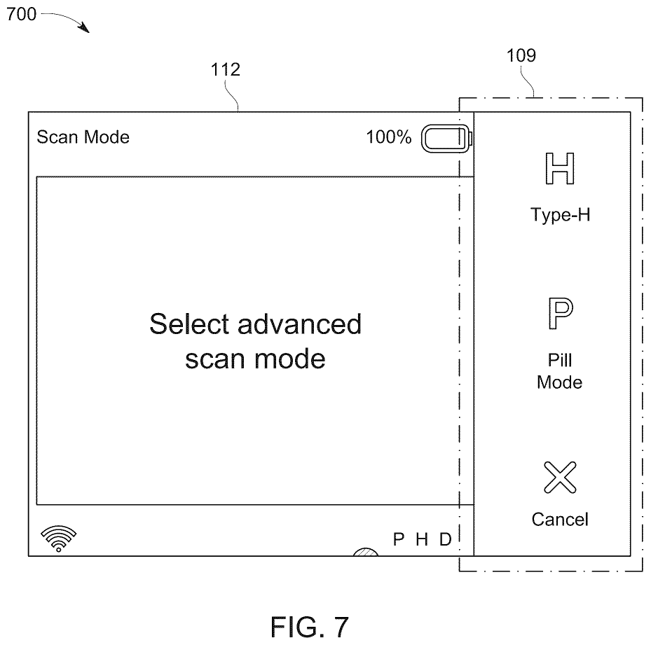

Referring to FIG. 7, a graphical user interface (GUI) 700 may be displayed on a display 112 of the spectroscopic system 110, where a user of the spectroscopic system 110 is prompted to make a selection of one or more scan modes through the I/O device 109, and the user selection is communicated to and received by the controller 111. The I/O device 109 also provides a cancel selection to revert to the previous page.

Referring back to FIG. 6, in operation 601, the controller 111 receives a first user selection input representative of a user selection of any one of one or more scan modes.

In some implementations, the controller 111 receives the first user selection input representative of one or more scan modes that may include a pill mode, a capsule mode, a natural form mode, a liquid mode, a loose powder mode, an injectable mode, or any combinations thereof. An example set of available scan modes, Type-H “H” and Pill mode “P”, are illustratively shown in FIG. 7.

In operation 603, in response to receiving the first user selection input, the controller 111 generates instructions for displaying to the user one or more selection options for any one of one or more target chemical substances to identify via a sample scan.

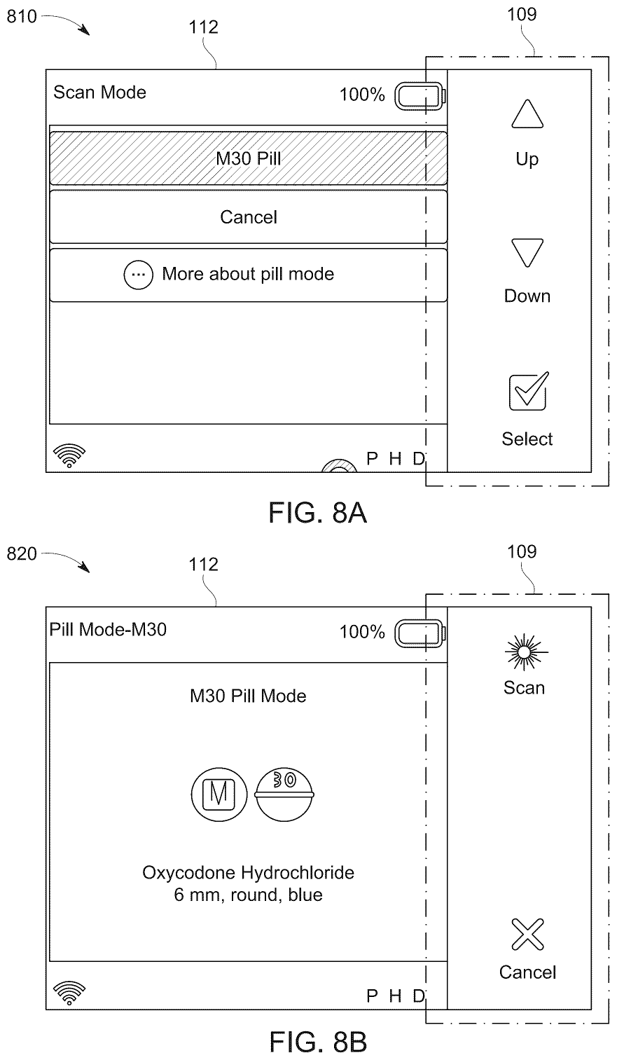

Referring to FIG. 8A, a graphical user interface (GUI) 810 is displayed on the display 112 of the spectroscopic system 110, where the user of the spectroscopic system 110 is prompted to make a second user selection of one of the one or more target chemical substances using I/O device 109. The second user selection of any one of the one or more target chemical substances is received by the controller 111. For illustrative purposes, the GUI 810 displays the target chemical substance “M30 Pill” option. In some implementations, GUI 810 displays one or more target chemical substances. The I/O device 109 also provides an up/down option for scrolling through the one or more options displayed on display 112 and a select option to confirm the selection of the one or more options displayed on display 112. The one or more options may include one or more target chemical substances, a “Cancel” option to revert to the previous page, and a “More about pill mode” to display indicia representative of a description of the Pill mode. For illustrative purposes, FIG. 8A illustrates the “M30 Pill,” the “Cancel”, and the “More about pill mode” options displayed on displayed 112.

In some implementations, the “M30 pill” option may be associated with an M30 pill having a chemical composition that includes oxycodone, one or more binders, one or more surfactants, one or more anti-caking agents, one or more coatings, one or more preservatives, one or more food-grade color dyes, one or more food-grade preservatives, or any combinations thereof. In some implementations, the M30 pill may include oxycodone between about 2.5 milligram (mg) to about 30 mg; about 5 mg; about 10 mg; about 15 mg; about 20 mg; about 25 mg; or about 30 mg.

Referring back to FIG. 6, in operation 605, the controller 111 receives the second user selection input representative of the user selection of any one of the one or more target chemical substances to identify. In some implementations, the one or more target chemical substances may include opioids, stimulants, depressants, designer drugs, hallucinogens, heroin, inhalants, cannabis, methamphetamine, steroids, variants, or any combinations thereof as described above.

Referring to FIG. 8B, a graphical user interface (GUI) 820 is displayed on the display 112 of the spectroscopic system 110, where the user of the spectroscopic system 110 is prompted to initiate the scan of the second user selection using the I/O device 109 (e.g., the selected target chemical substance). The I/O device 109 also includes a “Cancel” option to revert to the previous pill mode page, as illustratively depicted in FIG. 8A.

In operation 607, in response to receiving a second user selection input, the controller 111 generates instructions to initiate the light source 149 to scan the sample of the unknown chemical composition associated with the user selected target chemical substance. The light source 149 is communicatively connected to the analytical instrument support apparatus.

Referring back to FIG. 6, in some implementations, the controller 111 generates a set of program instructions and communicates the program instructions to activate the light source 149 to scan the sample of the unknown chemical composition.

In some implementations, the controller 111 generates a set of program instructions and communicates the set of program instructions to the display 112 to display a message. In some implementations, the message includes instructions to the user to direct (i.e., aim) the light source 149 toward the unknown chemical composition.

In some implementations, the controller 111 generates a set of program instructions and communicates the set of program instructions to the display 112 to display a hazard warning message. In some implementations, the hazard warning message includes instructions for safe handling of the spectroscopic system 110 and the sample of the unknown chemical composition. In some implementations, instructions for safe handling of the spectroscopic system 110 includes to avoid eye contact with the light source 149. In some implementations, instructions for safe handling of the unknown chemical composition includes wearing personal protective equipment (PPE) and to avoid contact with the unknown chemical composition while the user is not wearing PPE.

In some implementations, the controller 111 generates a set of program instructions and communicates the set of program instructions to the display 112 to display one or more operational instructions for proper use of the spectroscopic system 110. In some implementations, such as when the spectroscopic system 110 is a handheld or portable system, the one or more operational instructions include instructions for aiming the light source 149 (e.g., light emitted by the light source 149 and output by the system 110) toward the unknown chemical composition; positioning the system 110 (e.g., the light source 149) a specified distance from the unknown chemical composition; and maintaining the aiming and positioning of the system 110 until the result is determined based on the received scan data generated by the scan of the sample of the unknown chemical composition.

In some implementations, the controller 111 determines a low signal strength associated with a distance between light source 149 (e.g., the laser assembly 201 (FIG. 2) or the Raman laser 119 (FIG. 5)) and the sample of the unknown chemical composition. The controller 111 generates a set of program instructions and communications the set of program instructions to the display 112 to display a message to the user of the spectroscopic system 110 to reduce the distance between the light source 149 and the sample of the unknown chemical composition.

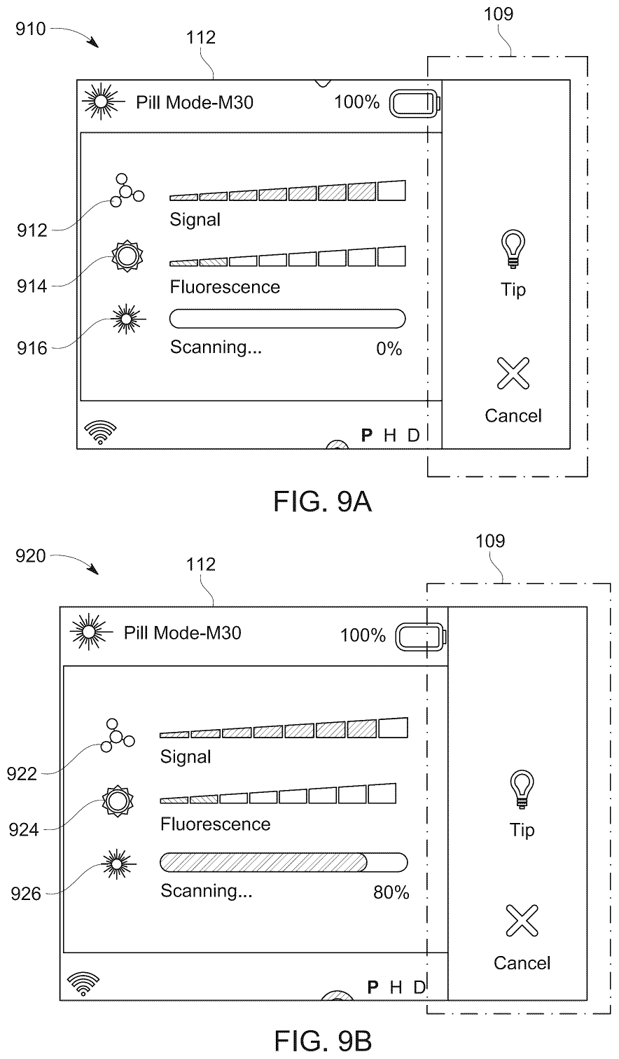

Referring to FIG. 9A, in some implementations, the controller 111 generates a set of program instructions and communicates the set of program instructions to the display 112 to display to the user of the spectroscopic system 110 (i) a signal strength 912, (ii) a fluorescence emission of the unknown chemical composition 914, and/or (iii) a percent complete of the scan 916. In some implementations, the signal strength 912 represents laser pulses being emitted from the light source 149 and directed toward the unknown chemical composition. In some implementations, the fluorescence emission is the fluorescence excitation of the unknown chemical composition from the light directed to the unknown chemical composition from the light source 149. In some implementations, the percent complete of the scan 916 includes either a percentage complete of the scan or a countdown timer representing the time until the scan is complete. In some implementations, the controller 111 generates one or more sets of program instructions and communicates the one or more sets of program instructions to the display 112 to display the one or more indicia indicating a percentage complete and/or a countdown timer until the scan of the unknown chemical composition is complete. FIG. 9A also depicts the I/O device 109 which includes a “Tip” option and a “cancel” option to cancel the scan and revert back to the previous page. The display 112 also depicts a red laser indicator in the top-left corner for when the light source 149 is on.

Referring to FIG. 9B, a continuation of the scan of the unknown chemical composition depicted in FIG. 9A is shown. FIG. 9B illustrates (i) the signal strength 922, (ii) the fluorescence emission of the unknown chemical composition 924, and/or (iii) the percent complete of the scan 926. FIG. 9A also depicts the I/O device 109 which includes a “Tip” option and a “cancel” option to cancel the scan and revert back to the previous page. The display 112 also depicts a red laser indicator in the top-left corner for when the light source 149 is on.

Referring back to FIG. 6, in operation 609, the controller 111 receives scan data generated by the scan of the sample of the unknown chemical composition. In some implementations, the received data may include data associated with the Raman measurement parameters.

In operation 611, the controller 111 determines a result based on the received scan data generated by the scan of the sample of the unknown chemical composition. In some implementations, the controller 111 determines the result based on at least one or more Raman measurement parameters.

In some implementations, the controller 111 analyzes the scan data generated by the scan of the sample of the unknown chemical composition. The controller 111 identifies the Raman measurement parameters, as discussed above, which may include the scan time and one or more Raman spectrums associated with the scan data. In some implementations, the controller 111 identifies one or more Raman shift wavenumbers associated with the one or more Raman spectrums.

In operation 613, in response to determining the result, the controller 111 compares the result against an expected result for a sample scan associated with the selected target chemical substance.

In some implementations, the controller 111 accesses a data storage device and retrieves the target chemical substance data stored on the data storage device. As discussed above, the target chemical substance data may include one or more known Raman signatures associated with the one or more target chemical substances including opioids, stimulants, depressants, designer drugs, hallucinogens, heroin, inhalants, cannabis, methamphetamine, steroids, synthetic variants, or any combinations thereof. In some implementations, the Raman signatures associated with the one or more target chemical substances may include the one or more Raman measurement parameters.

In some implementations, the comparison between the result and the expected result of the selected target chemical substance includes a determination of a similarity measure Si, as described above in equation (1).

In some implementations, the controller 111 determines that the result matches an expected result of the selected target chemical substance (e.g., target chemical substance data) (operation 615, YES branch). In some implementations the controller 111 determines a similarity measure greater than 95%, a match may be deemed to have occurred between the result and a Raman signature associated with the selected target chemical substance. In some implementations, the similarity measure is greater than 95%; greater than 96%; greater than 97%; greater than 98%; greater than 99%; or equal to 100%. In some implementations, the controller 111 determines a primary compound associated with the expected result of the selected target chemical substance.

For example, the controller 111 can determine that the primary compound, oxycodone, is associated with the expected result of the selected target chemical substance. The controller 111 can determine that the primary compound of the unknown chemical composition is oxycodone based on, at least, the result matching the Raman signature of the selected target chemical substance associated with the “M30 pill” option.

In operation 617, in response to the result based on the scan of the sample of the unknown chemical composition matching the expected result for the sample scan associated with the selected target chemical substance (e.g., the selected target chemical substance was detected), the controller 111 generates instructions for displaying indicia representative of a primary compound associated with the selected target chemical substance.

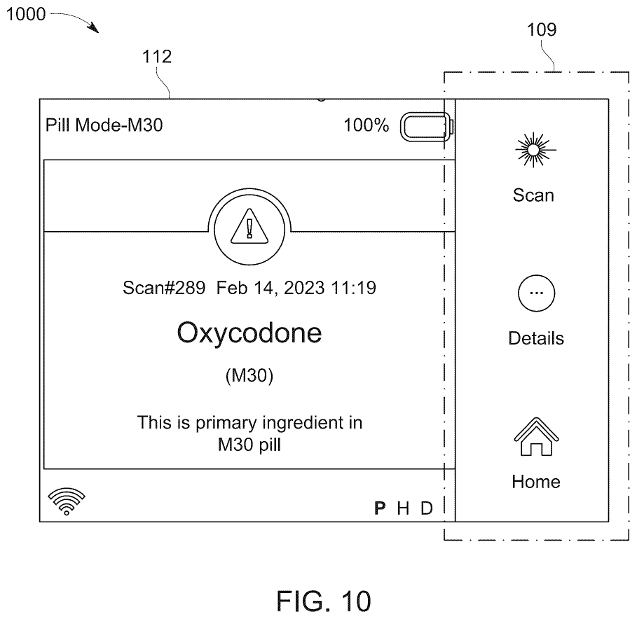

Referring to FIG. 10, a GUI 1000 is depicted that can be displayed on display 112 to the user of the spectroscopic system 110 (e.g., the Raman spectrometer 140) indicating that the primary compound associated with the selected target chemical substance. The user of spectroscopic system 110 is prompted to make one or more selection using the I/O device 109. The one or more selections includes (i) “Scan,” which will revert the display to the scan mode page, (ii) “Details,” which will display on display 112 details relating to the target chemical substance and additional information of the primary compound stored on target chemical substance data, and (iii) “Home,” which will revert the display to the home page. For illustrative simplicity, FIG. 10 shows that the primary compound is oxycodone, which is the primary compound for the selected target chemical substance, the M30 pill.

In some implementations, the controller 111 determines that the result does not match an expected result of the selected target chemical substance (e.g., selected target chemical substance is not detected) (operation 615, NO branch). In some implementations, the controller 111 determines a similarity measure of equal to or less than 95%, a match may not be deemed to have occurred between the result and the selected target chemical substance. In some implementations, the controller 111 does not determine a primary compound for the selected target chemical substance.

For example, the controller 111 can determine that the result does not match the Raman signature of the expected result associated with the selected target chemical substance. In such instances, the controller 111 determines that the result of the scan of the unknown chemical composition is inconclusive.

In operation 619, in response to the result based on the scan of the sample of the unknown chemical composition not matching the Raman signature of expected result associated with the selected target chemical substance, the controller 111 generates instructions for displaying indicia to the user representing that the result of the scan of the sample of the unknown chemical composition was inconclusive.

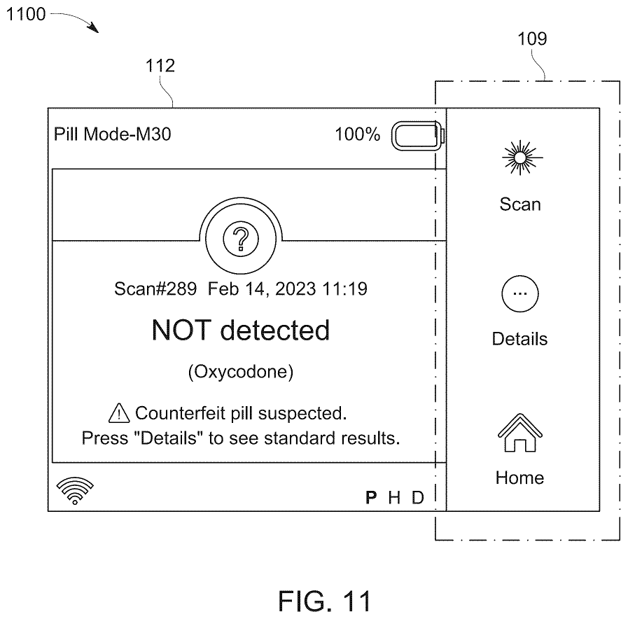

Referring to FIG. 11, a GUI 1100 is illustrated that can be displayed on the display 112 to the user of the spectroscopic system 110 that the result of the scan of the unknown chemical composition was inconclusive. The user of spectroscopic system 110 is prompted to make one or more selection using the I/O device 109. The one or more selections includes (i) “Scan,” which will revert the display to the scan mode page, (ii) “Details,” which will display on display 112 details relating to the target chemical substance and additional information of the primary compound stored on target chemical substance data, and (iii) “Home,” which will revert the display to the home page. For illustrative simplicity, FIG. 11 indicates that the primary compound of the expected result, oxycodone, could not be confirmed.

In some implementations, the inconclusive result, as described in operation 619, may occur when one or more exemplary counterfeit drug products are present in the unknown chemical composition or when one or more exemplary counterfeit drug products are not present in the unknown chemical composition.

In some implementations, the inconclusive result, as described in operation 619, may occur when an improper scan was completed, and improper scan data of the unknown chemical composition was received. In some implementations, at least, a second scan may be initiated using a proper scan.

The inconclusive result, as described in operation 619, may occur when the algorithm logic cannot come up with an identification result which satisfies certain thresholds of the algorithm logic, or due to a complexity of the mixture, or when a sample component is not part of the instrument's on-board library.

The inconclusive result, as described in operation 619, may occur when the sample does not give enough Raman signal in the allotted scan time to satisfy SNR thresholds. This may be due to high sample fluorescence, the color of the sample, a poor Raman scatterer, the sample and instrument not being positioned properly relative to each other, or a malfunctioning instrument.

In some implementations, the inconclusive result, as described in operation 619, may indicate an environmental parameter impacted the scan of the unknown chemical composition, and improper scan data of the unknown chemical composition was received. In some implementations, at least, a second scan may be initiated.

In some implementations, the inconclusive result, as described in operation 619, may indicate that the spectroscopic system 110 is not calibrated properly, and improper scan data of the unknown chemical composition was received. In some implementations, at least, a second scan may be initiated, such as after the spectrometer is properly calibrated.

As described above in the detailed description, reference is made to the accompanying drawings that form a part hereof wherein like numerals designate like parts throughout, and in which is shown, by way of illustration, implementations that may be practiced. It is to be understood that other implementations may be utilized, and structured or logical changes may be made, without departing from the scope of the present disclosure. Therefore, the detailed description as described above is not to be taken in a limiting sense.

Various operations may be described as multiple discrete actions or operations in turn, in a manner that is most helpful in understanding the subject matter disclosed herein. However, the order of description should be construed as to imply that these operations are necessarily order dependent. In particular, these operations may not be performed in the order of presentation. Operations described may be performed in a different order from the described implementation. Various additional operations may be performed, and/or described operations may be omitted in additional implementations.

### Clauses

Implementations of the present disclosure are disclosed in the following clauses:

Clause 1. A computer-implemented method on an analytical instrument support apparatus, the method comprising: receiving, by one or more processors, a first user selection input representative of a user selection of any one of one or more scan modes; in response to receiving the first user selection input, generating instructions by one or more processors for displaying to the user one or more selection options for any one of one or more target chemical substances to identify via a sample scan; receiving, by one or more processors, a second user selection input representative of a user selection of any one of the one or more target chemical substances to identify; in response to receiving the second user selection input, generating instructions by one or more processors for initiating a laser to scan a sample of an unknown chemical composition, the laser being communicatively connected to the analytical instrument support apparatus; receiving, by one or more processors, scan data generated by a scan of the sample of the unknown chemical composition; determining, by one on or more processors, a result based on the received scan data generated by the scan of the sample of the unknown chemical composition; in response to determining the result, comparing, by one or more processors, the result against an expected result for a sample scan associated with the selected target chemical substance; in response to the result based on the scan of the sample of the unknown chemical composition matching the expected result for the sample scan associated with the selected target chemical substance, generating instructions for displaying, by one or more processors, indicia representative of a primary compound associated with the selected target chemical substance; and in response to the result based on the scan of the sample of the unknown chemical composition not matching the expected result for the sample scan associated with the selected target chemical substance, generating instructions for displaying, by one or more processors, indicia to the user representing that the scan of the sample of the unknown chemical composition result was inconclusive.

Clause 2. The computer-implemented method of clause 1, wherein the laser is a Raman laser.

Clause 3. The computer-implemented method of clause 2, wherein the user is instructed to aim light emitted by the Raman laser toward the sample of the unknown chemical composition.

Clause 4. The computer-implemented method of clause 1, wherein the scan mode includes pill mode, capsule mode, natural form mode, liquid mode, loose powder mode, or injectable mode.

Clause 5. The computer-implemented method of clause 1, wherein the one or more target chemical substances include opioids, stimulants, depressants, designer drugs, hallucinogens, heroin, inhalants, cannabis, methamphetamine, steroids, synthetic variants, or any combinations thereof.

Clause 6. The computer-implemented method of clause 1, the method further comprising generating instructions by one or more processors for displaying one or more safety instructions for handling of (i) the analytical instrument support apparatus and (ii) the sample of the unknown chemical composition.

Clause 7. The computer-implemented method of clause 2, the method further comprising: generating instructions by one or more processors for displaying a signal strength associated with the Raman laser and the sample of the unknown chemical composition; determining, by one or more processors, a low signal strength associated with a distance between the Raman laser and the sample of the unknown chemical composition; and generating instructions by one or more processors for displaying to the user of the analytical instrument support apparatus a message to reduce the distance between the Raman laser and the sample of the unknown chemical composition.

Clause 8. The computer-implemented method of clause 1, the method further comprising: in response to generating instructions for initiating the laser to scan, generating instructions by one or more processors for displaying one or more messages indicating a percentage complete of the scan of the unknown chemical composition.

Clause 9. The computer-implemented method of clause 1, wherein the primary compound is a pharmaceutical compound.

Clause 10. The computer-implemented method of clause 1, wherein the determination of the result of the scan of the unknown chemical composition further comprises: determining, by one or more processors, that a primary compound associated with the unknown chemical composition is not detected; and generating instructions by one or more processors to display indicia to the user of the analytical instrument support apparatus representing that the sample of the unknown chemical composition may include one or more counterfeit drug products.

Clause 11. The computer-implemented method of clause 1, wherein the determination of the result of the scan of a sample of the unknown chemical composition further comprises: determining, by one or more processors, that an opioid substance associated with the selected target chemical substance is not detected, wherein the opioid substance is oxycodone; and generating instructions by one or more processors to display indicia to the user of the analytical instrument support apparatus representing that the sample of the unknown chemical composition may include fentanyl.

Clause 12. The computer-implemented method of clause 1, wherein a match between the result and the expected result for the sample scan associated with the selected target chemical substance is based on a similarity measure of greater than 95%.

Clause 13. The computer-implemented method of clause 1, the method further comprising displaying, by one or more processors, one or more operational instructions to the user of the analytical instrument support apparatus to operate the analytical instrument support apparatus, the one or more operational instructions including instructions for: aiming light emitted by the laser toward the unknown chemical composition; positioning the laser a specified distance from the unknown chemical composition; and maintaining the aiming and positioning until the result is determined based on the received scan data generated by the scan of the sample of the unknown chemical composition.

Clause 14. One or more non-transitory computer-readable media having instructions stored thereon that, when executed by one or more processing devices of an analytical instrument support apparatus, cause the analytical instrument support apparatus to perform the computer-implemented method of clause 1.

Clause 15. An analytical instrument support system comprising: one or more processors, one or more non-transitory computer-readable storage media; and program instructions stored on at least one of the one or more non-transitory computer-readable storage media for execution by at least one of the one or more processors, the program instructions comprising: program instructions to receive a first user selection input representative of a user selection of any one of one or more scan modes; in response to receiving the first user selection input, program instructions to generate instructions for displaying to the user one or more selection options for any one of one or more target chemical substances to identify via a sample scan; program instructions to receive a second user selection input representative of a user selection of any one of the one or more target chemical substances to identify; in response to receiving the second user selection input, program instructions to generate instructions for initiating a laser to scan a sample of an unknown chemical composition, the laser being communicatively connected to the analytical instrument support system; program instructions to receive scan data generated by a scan of the sample of the unknown chemical composition; program instructions to determine a result based on the received scan data generated by the scan of the sample of the unknown chemical composition; in response to determining the result, program instructions to compare the result against an expected result for a sample scan associated with the selected target chemical substance; in response to the result based on the scan of the sample of the unknown chemical composition matching the expected result for the sample scan associated with the selected target chemical substance, program instructions to generate instructions for displaying indicia representative of a primary compound associated with the selected target chemical substance; and in response to the result based on the scan of the sample of the unknown chemical composition not matching the expected result for the sample scan associated with the selected target chemical substance, program instructions to generate instructions for displaying indicia to the user representing that the scan of the sample of the unknown chemical composition result was inconclusive.

Clause 16. The analytical instrument support system of clause 15, wherein the program instructions are executed on a common computing device including at least one of the one or more processors.

Clause 17. The analytical instrument support system according to any one of clauses 15 or 16, wherein the program instructions are executed on a computing device including at least one of the one or more processors, and wherein the computing device is remote from an analytical instrument associated with the analytical instrument support system.

Clause 18. The analytical instrument support system according to any one of clauses 15-17, wherein the program instructions are executed on a user computing device including at least one of the one or more processors.

Clause 19. The analytical instrument support system according to any one of clauses 15-18, wherein at least one of the one or more processors is included in an analytical instrument associated with the analytical instrument support system, and wherein the program instructions are executed on the at least one of the one or more processors.

Clause 20. An analytical instrument comprising: a light source configured to emit light toward a surface of a sample; a spectrometer configured to acquire a Raman spectrum from scattered light produced by the sample in response to the light emitted by the light source; one or more processors; one or more non-transitory computer-readable storage media; and program instructions stored on at least one of the one or more non-transitory computer-readable storage media for execution by at least one of the one or more processors, wherein execution of the program instructions by at least one of the one or more processors cause the analytical instrument to implement a set of functions comprising: receiving a first user selection input representative of a user selection of any one of one or more scan modes; in response to receiving the first user selection input, program instructions to generate instructions for displaying to the user one or more selection options for any one of one or more target chemical substances to identify via a sample scan; program instructions to receive a second user selection input representative of a user selection of any one of the one or more target chemical substances to identify; in response to receiving the second user selection input, program instructions to generate instructions for initiating a laser to scan a sample of an unknown chemical composition, the laser being communicatively connected to the analytical instrument; program instructions to receive scan data generated by the scan of the sample of the unknown chemical substance; program instructions to determine a result based on the received scan data generated by a scan of the sample of the unknown chemical composition; in response to determining the result, program instructions to compare the result against an expected result for a sample scan associated with the selected target chemical substance; in response to the result based on the scan of the sample of the unknown chemical composition matching the expected result for the sample scan associated with the selected target chemical substance, program instructions to generate instructions for displaying indicia representative of a primary compound associated with the selected target chemical substance; and in response to the result based on the scan of the sample of the unknown chemical composition not matching the expected result for the sample scan associated with the selected target chemical substance, program instructions to generate instructions for displaying indicia to the user representing that the scan of the sample of the unknown chemical composition result was inconclusive.

Clause 21. The analytical instrument according to clause 20, wherein the light source is a Raman laser.

Clause 22. The analytical instrument according to clause 21, wherein the user is instructed to aim light emitted by the Raman laser toward the sample of the unknown chemical composition.

Clause 23. The analytical instrument according to clause 20, wherein the scan mode includes pill mode, capsule mode, natural form mode, liquid mode, loose powder mode, or injectable mode.

Clause 24. The analytical instrument according to clause 20, wherein the one or more target chemical substances include opioids, stimulants, depressants, designer drugs, hallucinogens, heroin, inhalants, cannabis, methamphetamine, steroids, synthetic variants, and any combinations thereof.

Clause 25. The analytical instrument according to clause 20, further comprising: generating instructions by one or more processors for displaying one or more safety instructions for handling of (i) the analytical instrument and (ii) the sample of the unknown chemical composition.

Clause 26. The analytical instrument according to clause 21, further comprising: generating instructions by one or more processors for displaying a signal strength associated with the Raman laser and the sample of the unknown chemical composition; determining, by one or more processors, a low signal strength associated with a distance between the Raman laser and the sample of the unknown chemical composition; and generating instructions by one or more processors for displaying to the user of the analytical instrument a message to reduce the distance between the Raman laser and the sample of the unknown chemical composition.

Clause 27. The analytical instrument according to clause 20, further comprising: in response to generating instructions for initiating the laser to scan, generating instructions by one or more processors for displaying one or more messages indicating a percentage complete of the scan of the unknown chemical composition.

Clause 28. The analytical instrument according to clause 20, wherein the primary compound is a pharmaceutical compound.

Clause 29. The analytical instrument according to clause 20, wherein the determination of the result of the scan of the unknown chemical substance further comprises: determining, by one or more processors, that a primary compound associated with the unknown chemical composition is not detected; and generating instruction by one or more processors to display indicia to the user of the analytical instrument representing that the sample of the unknown chemical composition may include one or more counterfeit drug products.

Clause 30. The analytical instrument according to clause 20, wherein the determination of the result of the scan of a sample of an opioid substance further comprises: determining, by one or more processors, that an opioid substance associated with the selected target chemical substance is not detected, wherein the opioid substance is oxycodone; and generating instructions by one or more processors to display indicia to the user of the analytical instrument representing that the sample of the unknown chemical composition may include fentanyl.

Clause 31. The analytical instrument according to clause 20, wherein a match between the results and the expected result for the sample scan associated with the selected target chemical substance is based on a similarity measure of greater than 95%.

Clause 32. The analytical instrument according to clause 20, further comprising: displaying, by one or more processors, one or more operational instructions to the user of the analytical instrument to operate the analytical instrument, the one or more operational instructions including instructions for: aiming the laser toward the unknown chemical composition; positioning the laser a specified distance from the unknown chemical composition; and maintaining the aiming and positioning until the result is determined based on the received scan data generated by the scan of the sample of the unknown chemical composition.

What is claimed is:

1. A computer-implemented method on an analytical instrument support apparatus, the method comprising:
receiving, by one or more processors, a first user selection input representative of a user selection of any one of one or more scan modes;
in response to receiving the first user selection input, generating instructions by one or more processors for displaying to the user one or more selection options for any one of one or more target chemical substances to identify via a sample scan;
receiving, by one or more processors, a second user selection input representative of a user selection of any one of the one or more target chemical substances to identify;
in response to receiving the second user selection input, generating instructions by one or more processors for initiating a laser to scan a sample of an unknown chemical composition, the laser being communicatively connected to the analytical instrument support apparatus;
receiving, by one or more processors, scan data generated by a scan of the sample of the unknown chemical composition;
determining, by one on or more processors, a result based on the received scan data generated by the scan of the sample of the unknown chemical composition;
in response to determining the result, comparing, by one or more processors, the result against an expected result for a sample scan associated with the selected target chemical substance; and
in response to the result based on the scan of the sample of the unknown chemical composition matching the expected result for the sample scan associated with the selected target chemical substance, generating instructions for displaying, by one or more processors, indicia representative of a primary compound associated with the selected target chemical substance.

2. The computer-implemented method of claim 1, wherein the laser is a Raman laser and wherein the user is instructed to aim light emitted by the Raman laser toward the sample of the unknown chemical composition.

3. The computer-implemented method of claim 2, the method further comprising:
generating instructions by one or more processors for displaying a signal strength associated with the Raman laser and the sample of the unknown chemical composition;
determining, by one or more processors, a low signal strength associated with a distance between the Raman laser and the sample of the unknown chemical composition; and
generating instructions by one or more processors for displaying to the user of the analytical instrument support apparatus a message to reduce the distance between the Raman laser and the sample of the unknown chemical composition.

4. The computer-implemented method of claim 1, wherein the scan mode includes pill mode, capsule mode, natural form mode, liquid mode, loose powder mode, or injectable mode.

5. The computer-implemented method of claim 1, wherein the one or more target chemical substances include opioids, stimulants, depressants, designer drugs, hallucinogens, heroin, inhalants, cannabis, methamphetamine, steroids, synthetic variants, or any combinations thereof.

6. The computer-implemented method of claim 1, the method further comprising generating instructions by one or more processors for displaying one or more safety instructions for handling of (i) the analytical instrument support apparatus and (ii) the sample of the unknown chemical composition.

7. The computer-implemented method of claim 1, wherein the primary compound is a pharmaceutical compound.

8. The computer-implemented method of claim 1, wherein the determination of the result of the scan of the unknown chemical composition further comprises:
determining, by one or more processors, that a primary compound associated with the unknown chemical composition is not detected; and
generating instructions by one or more processors to display indicia to the user of the analytical instrument support apparatus representing that the sample of the unknown chemical composition may include one or more counterfeit drug products.

9. The computer-implemented method of claim 1, wherein the determination of the result of the scan of a sample of the unknown chemical composition further comprises:
determining, by one or more processors, that an opioid substance associated with the selected target chemical substance is not detected,
wherein the opioid substance is oxycodone; and
generating instructions by one or more processors to display indicia to the user of the analytical instrument support apparatus representing that the sample of the unknown chemical composition may include fentanyl.

10. An analytical instrument comprising:
a light source configured to emit light toward a surface of a sample;
a spectrometer configured to acquire a Raman spectrum from scattered light produced by the sample in response to the light emitted by the light source;
one or more processors;
one or more non-transitory computer-readable storage media; and
program instructions stored on at least one of the one or more non-transitory computer-readable storage media for execution by at least one of the one or more processors, wherein execution of the program instructions by at least one of the one or more processors cause the analytical instrument to implement a set of functions comprising:
receiving a first user selection input representative of a user selection of any one of one or more scan modes;
in response to receiving the first user selection input, program instructions to generate instructions for displaying to the user one or more selection options for any one of one or more target chemical substances to identify via a sample scan;
program instructions to receive a second user selection input representative of a user selection of any one of the one or more target chemical substances to identify;
in response to receiving the second user selection input, program instructions to generate instructions for initiating a laser to scan a sample of an unknown chemical composition, the laser being communicatively connected to the analytical instrument;
program instructions to receive scan data generated by the scan of the sample of the unknown chemical substance;
program instructions to determine a result based on the received scan data generated by a scan of the sample of the unknown chemical composition;
in response to determining the result, program instructions to compare the result against an expected result for a sample scan associated with the selected target chemical substance;
in response to the result based on the scan of the sample of the unknown chemical composition matching the expected result for the sample scan associated with the selected target chemical substance, program instructions to generate instructions for displaying indicia representative of a primary compound associated with the selected target chemical substance; and
in response to the result based on the scan of the sample of the unknown chemical composition not matching the expected result for the sample scan associated with the selected target chemical substance, program instructions to generate instructions for displaying indicia to the user representing that the scan of the sample of the unknown chemical composition result was inconclusive.

11. The analytical instrument according to claim 10, wherein the light source is a Raman laser and wherein the user is instructed to aim light emitted by the Raman laser toward the sample of the unknown chemical composition.

12. The analytical instrument according to claim 11, further comprising:
generating instructions by one or more processors for displaying a signal strength associated with the Raman laser and the sample of the unknown chemical composition;
determining, by one or more processors, a low signal strength associated with a distance between the Raman laser and the sample of the unknown chemical composition; and
generating instructions by one or more processors for displaying to the user of the analytical instrument a message to reduce the distance between the Raman laser and the sample of the unknown chemical composition.

13. The analytical instrument according to claim 10, wherein the scan mode includes pill mode, capsule mode, natural form mode, liquid mode, loose powder mode, or injectable mode.

14. The analytical instrument according to claim 10, wherein the one or more target chemical substances include opioids, stimulants, depressants, designer drugs, hallucinogens, heroin, inhalants, cannabis, methamphetamine, steroids, synthetic variants, and any combinations thereof.

15. The analytical instrument according to claim 10, wherein the primary compound is a pharmaceutical compound.

16. The analytical instrument according to claim 10, wherein the determination of the result of the scan of the unknown chemical substance further comprises:
determining, by one or more processors, that a primary compound associated with the unknown chemical composition is not detected; and
generating instruction by one or more processors to display indicia to the user of the analytical instrument representing that the sample of the unknown chemical composition may include one or more counterfeit drug products.

17. The analytical instrument according to claim 10, wherein the determination of the result of the scan of a sample of an opioid substance further comprises:
determining, by one or more processors, that an opioid substance associated with the selected target chemical substance is not detected,
wherein the opioid substance is oxycodone; and
generating instructions by one or more processors to display indicia to the user of the analytical instrument representing that the sample of the unknown chemical composition may include fentanyl.

18. The analytical instrument according to claim 10, wherein a match between the results and the expected result for the sample scan associated with the selected target chemical substance is based on a similarity measure of greater than 95%.

19. The analytical instrument according to claim 10, further comprising:
displaying, by one or more processors, one or more operational instructions to the user of the analytical instrument to operate the analytical instrument, the one or more operational instructions including instructions for:
aiming the laser toward the unknown chemical composition;
positioning the laser a specified distance from the unknown chemical composition; and
maintaining the aiming and positioning until the result is determined based on the received scan data generated by the scan of the sample of the unknown chemical composition.

20. One or more non-transitory computer-readable media having instructions stored thereon that, when executed by one or more processing devices of an analytical instrument support apparatus, cause the analytical instrument support apparatus to perform a set of functions comprising:
receiving a first user selection input representative of a user selection of any one of one or more scan modes;
in response to receiving the first user selection input, generating instructions for displaying to the user one or more selection options for any one of one or more target chemical substances to identify via a sample scan;
receiving a second user selection input representative of a user selection of any one of the one or more target chemical substances to identify;
in response to receiving the second user selection input, generating instructions for initiating a laser to scan a sample of an unknown chemical composition, the laser being communicatively connected to the analytical instrument support apparatus;
receiving scan data generated by a scan of the sample of the unknown chemical composition;
determining a result based on the received scan data generated by the scan of the sample of the unknown chemical composition;
in response to determining the result, comparing the result against an expected result for a sample scan associated with the selected target chemical substance;
in response to the result based on the scan of the sample of the unknown chemical composition matching the expected result for the sample scan associated with the selected target chemical substance, generating instructions for displaying indicia representative of a primary compound associated with the selected target chemical substance; and
in response to the result based on the scan of the sample of the unknown chemical composition not matching the expected result for the sample scan associated with the selected target chemical substance, generating instructions for displaying indicia to the user representing that the scan of the sample of the unknown chemical composition result was inconclusive.
